(19) United States
(12) Patent Application Publication
(10) Pub. No.: US 2012/0329872 A1
(43) Pub. Date: Dec. 27, 2012

(54) Ready-To-Eat Feed For Domestic Pets

(75) Inventors: Thomas Gastner, Engelsberg (DE); Hans-Peter Krimmer, Kirchweidach (DE)

(73) Assignee: Alzchem Trostberg GmbH, Trostberg (DE)

(21) Appl. No.: 13/539,518

(22) Filed: Jul. 2, 2012

Related U.S. Application Data
(63) Continuation of application No. 12/281,015, filed on Nov. 18, 2008, now abandoned.

(30) Foreign Application Priority Data
Mar. 1, 2006 (DE) 10 2006 009 373.9

(51) Int. Cl. A61K 31/198; A61P 3/02; A61K 31/205

(52) U.S. Cl. 514/556; 514/565

(57) ABSTRACT

A new finished feed for domestic pets is proposed, containing at least one guanidinoacetic acid component as the active component with regard to nutritional physiology. The new finished feed which preferably has a water content of >8% by weight, can be produced in an extremely economical manner, wherein the main component has a significantly higher stability during passage through the gastrointestinal tract and is therefore only converted into creative under physiological conditions. For this reason the guanidinoacetic acid is also utilized to a high degree by the target group which is in particular cats and dogs.

### PRIOR RELATED APPLICATIONS

This application is a continuation of U.S. patent application Ser. No. 12/281,015 filed Nov. 18, 2008, which is a national phase of PCT/EP2007/001783 filed Mar. 1, 2007, which claims the benefit of priority to DE 10 2006 009 373.9 filed Mar. 1, 2006, each of which is incorporated by reference herein in its entirety.

### BRIEF DESCRIPTION OF THE DRAWINGS

FIG. 1. Content of creatine and guanidinoacetic acid in water at pH 7 following exposure to 160° C. for 30 minutes. Guanidinoacetic acid has significantly higher stability than creatine.

FIG. 2. Content of creatine and guanidinoacetic acid in water at pH 5 over 60 days. Guanidinoacetic acid has significantly higher storage stability than creatine.

### DESCRIPTION

The present invention concerns a finished feed for domestic pets which contains a guanidinoacetic acid component as the active component in terms of nutritional physiology.

Guanidinoacetic acid (GAA) is an endogenous substance which occurs in animals and also in humans and plays a central role in the biosynthesis of creatine. Creatine can be assimilated from food and also be formed endogenously. The biosynthesis starts from glycine and L-arginine. In mammals the guanidino group of L-arginine is cleaved and an N-C-N group is transferred to glycine by the enzyme aminotransferase primarily in the kidneys but also in the liver and pancreas. L-arginine is converted into L-ornithine in this process. The guanidinoacetic acid that is formed in this manner is converted in the next step into creatine with the aid of the enzyme transmethylase which occurs exclusively in the liver in the case of vertebrates. In this process S-adenosyl methionine serves as a methyl group donor. The creatine is subsequently transported via the blood circulation to the target organs. It is transported through the cell membrane into the cells by a specific creatine transporter.

Several working groups have already shown in clinical studies in the fifties of the last century that the administration of guanidinoacetic acid in combination with betaine has a positive effect on the course of the disease in the case of cardiac diseases. The patients reported a considerable improvement in their general state of health. In addition an improved endurance during physical exercise and increased muscle strength were already found after a short treatment period. The patients also reported an improved libido. 200 patients were administered a dose of 30 mg GAA/kg daily for one year. Side effects were not observed (Borsook H.; Borsook M. E.: The biochemical basis of betaine-glycocyamine therapy. In Annals of western medicine and surgery 5(10), 825, 1951).

The International Patent Application WO 91/07954 A1 discloses the use of guanidinoacetic acid in combination with methionine or S-adenosyl methionine to increase the creatine level in the muscle. Conditions were mentioned as a field of application which require an increased creatine level in the muscle. Medical applications as well as the field of sport nutrition are claimed.

In this connection it is asserted that the administration of creative does not increase the creatine level. This assertion has now been disproven by numerous publications (e.g. Persky, A. M., Brazeau, G. A.: Clinical Pharmacology of the Dietary Supplement Creatine Monohydrate. In: Pharmacol. Rev. 2001, 53, 161-176). A direct comparison of the efficacy of creatine and guanidinoacetic acid is not disclosed in WO 91/07954.

It is also known that guanidinoacetic acid has an antibacterial action and has been successfully used against bacterial infections (Staphylococcus aureus) in animal experiments (Preparation for protecting mammals against infection, Stanley Drug Products Inc. USA; Neth. Appl. (1976), 7 pp. NL 7411216).

In connection with the overdosing of methionine it is also known that the associated negative effects can be attenuated by the administration of guanidinoacetic acid (Interrelations of choline and methionine in growth and the action of betaine in replacing them. McKittrick, D. S., Univ. of California, Berkeley, Archives of Biochemistry (1947), 15, 133-155).

The International Patent Application WO 2004/000297 A1 describes a mixture for feeding or pharmaceutical purposes in mammals. This mixture consists of a protein fraction which contains L-serine and guanidinoacetic acid as a further component. The mixture should in this connection be free from glycine or, after hydrolysis of the mixture, it should contain a ratio of L-serine to glycine of more than 2.7:1. Solutions, emulsions, suspensions, gels, bars, sweets and preferably powder are mentioned as possible forms of the product. There is no mention of the use of guanidinoacetic acid as a finished feed for domestic animals.

A ratio of L-serine to glycine of more than 2.7:1 is not encountered in commercially available pet food for domestic pets. Raw materials from animals such as e.g. meat and bone meal contain considerably more glycine than serine (Amino acids of meals of animal origin; de Vuyst, A. Univ. Louvain, Belgium, Agricultura (Heverlee, Belgium) (1964), 12(1), 141-151). In plant raw materials the ratio between glycine and serine is predominantly balanced.

Creatine plays an important role in the energy metabolism of the cell where in addition to adenosine triphosphate (ATP), it represents an important energy reserve of the muscle in the fowl of energy-rich phosphocreatine. In the resting state of the muscle ATP can transfer a phosphate group onto creatine to form phosphocreatine which is then in direct equilibrium with ATP. During muscle work it is very important to fill up the ATP stores again as rapidly as possible. Phosphocreatine is available for this purpose in the first seconds of maximum muscle load. A phosphate group can be transferred from phosphocreatine onto adenosine diphosphate by the enzyme creatine kinase in a very rapid reaction and thus regenerate ATP. This is also referred to as the Lohmann reaction.

Creatine has been known for a long time as a suitable food supplement and animal feed. The creatine stores that are naturally present in the body are rapidly exhausted during intense and prolonged muscle work. Targeted administration of creatine has a positive effect on the endurance and performance especially in competitive athletes where undesired accumulation processes in the body or disadvantageous degradation products are unknown. The reason for this is because if creatine is fed in excess, it is eliminated from the body as creatine and creatiniue.

Furthermore, it is known that a creatine supplementation results in an increase of body mass. This is initially due to an increased uptake of water into the muscle. However, in the long-term creatine indirectly results in an increase in muscle mass due to increased protein synthesis or a reduced protein catabolism in the myofibrils. (Int. J. Sports Med. 21 (2000), 139-145). Thus, the result is an increased fat-free body mass.

In addition to creatine itself i.e. creatine monohydrate, numerous creatine salts such as creatine ascorbate, citrate, pyruvate and others have in the meantime also proven to be suitable food supplements. At this point the European Patent EP 894 083 B1 and the German laid-open patent application DE 197 07 694 A1 are mentioned as representatives.

The proven positive effects in humans are also displayed by creatine in animals which is why its use in diverse animal feeds is also sufficiently previously described. Studies on dogs were already carried out by Benedict and Osterberg in 1923. It was observed that creatine administered orally at a daily dose of about 40 mg/kg for several weeks results in a considerable increase in weight. A positive nitrogen balance was also observed (The Journal of Biological Chemistry No. 1 (1923), 229-252).

GB 2 300 103 teaches the use of creatine in the form of a dog biscuit for which purpose creatine monohydrate is offered together with meat in an extruded paste.

The use of creatine or creatine salts as a feed additive for breeding animals and fattened animals, as a substitute for meat and bone meal, fish meal and/or antimicrobial performance enhancers, growth hormones and anabolics has been previously described in the International Patent Application WO 00/67 590 A1.

Since creatine monohydrate is insufficiently bioavailable due to its poor solubility, it is recommended that is should be used together with other physiologically active compounds preferably in a salt form. The German laid-open specification DE 198 450 A1 concerns the use of stable pyruvic acid salts in particular of creatine pyruvate in formulations which are suitable for animal feeds.

Creatine is a natural component in the diet of carnivorous and omnivorous wild animals. Thus wolves which have a body weight between 15 and 60 kg eat on average 100-130 g meat per kilogram body weight per day. Fresh meat contains between 3 and 6 g (23-46 mmol) creatine per kilogram. Thus, a wolf of 35 kg takes in about 3.5 to 4.5 kg fresh meat which contains between 10.5 and 27 g creatine. In contrast about 1.25 kg meat is sufficient for domesticated dogs with a body weight of 35 kg. If it is ingested in a fresh and raw form, it contains between 3.75 and 7.5 g creatine (Research in Veterinary Science 62 (1997), 58-62).

In addition to its undisputed positive physiological properties, creatine, however, also has the disadvantage that it is very unstable in aqueous solutions and moist formulations especially at high temperatures where it is converted into creatinine. Commercially produced animal feed is heated strongly during processing to make it stable. Thus, for example in the production of dry dog and cat biscuits, the raw materials are heated in extruders to temperatures of up to 190° C. Humidity, pressure and heat gelatinize the starch that is present and the paste that is obtained is subsequently brought into the desired form. The high temperatures during processing and the storage under moist conditions such as for example in canned food which contains about 75-85% water has the effect that most of the creatine that it contains is converted into creatinine. This was also demonstrated by Harris in commercial canned food and dry food for dogs. The examined eight canned foods only contained traces of creatine (0.36 to 1.93 mmol/kg). Also in dry feeds values of 0.7 mmol creatine per kilogram were measured in most of the samples (Research in Veterinary Science 62 (1997), 58-62). Thus, it is apparent that dogs and cats which are fed with commercial animal feeds (0.36-4.25 mmol creatine per kilogram feeding stuff) ingest considerably less creatine via the food than would be the case with a natural diet with fresh meat (23-46 mmol creatine per kilogram).

This instability of creatine is also important with respect to oral ingestion. The pH of the stomach of 1 to 2 can result in a considerable degradation of creatine to creatinine depending on the retention time. Thus, in humans it was shown that after an oral administration of creatine, only about 15 to 30% could be reabsorbed by the muscle (Greenhaff, P. L.: Factors Modifying Creatine Accumulation in Human Skeletal Muscle. In: Creatine. From Basic Science to Clinical Application. Medical Science Symposia Series Volume 14, 2000, 75-82).

From the described disadvantages of the state of the art with regard to creatine, the object of the present invention was to find compounds for finished feeds which, if possible, have a low instability in industrial processing processes. They should not only withstand high processing temperatures without damage, but should also be stable when stored for example in canned feeds under moist conditions. Furthermore, the compound, in contrast to creatine, should survive the acidic environment of the stomach without damage and not be converted into creatine until it has been taken up into the body. The feed additives that are used should themselves display no physiologically disadvantageous effects and should be easy to detect. From an economic point of view it is important that the substances that are used according to the invention can be produced in an economically favourable manner.

This object was achieved by finished feeds for domestic pets which contain guanidinoacetic acid and/or guanidinoacetic acid salts as the active component with regard to nutritional physiology.

In finished feeds it was surprisingly found that the guanidinoacetic acid components do in fact fulfill the requirement profile according to the object because they can be produced in a simple and economic manner; in contrast to creatine or creatine monohydrate, guanidinoacetic acid and salts thereof have a considerably higher stability in acidic solutions such as those that occur in the stomach and they are only converted into creatine under physiological conditions. Surprisingly it has turned out to be particularly advantageous that in contrast to creatine, guanidinoacetic acid and salts thereof described in the present connection are thus not converted until after they have been reabsorbed which occurs primarily in the liver. Thus, in contrast to the known creatine most of the compounds used are not already degraded in advance by instability reactions and eliminated, but are in fact made available to the physiological fields of application. Thus, according to the invention guanidinoacetic acid and salts thereof can be used in considerably lower dosages compared to creatine, while having an identical effect.

Furthermore, it was possible to show that guanidinoacetic acid has a very high stability under conditions such as those which occur during the industrial production of feedstuffs. In this connection guanidinoacetic acid exhibits clear advantages over creatine. In addition it was possible to show that guanidinoacetic acid has a considerably better storage stability than creatine. These advantages were thus in their entirety not predictable.

Due to the surprisingly favourable properties of the guanidinoacetic acid component in the claimed finished feed, it is not limited to specific forms of administration. But rather variants in the form of dry, semi-moist and wet feeds come equally into consideration such as in particular canned feeds, pellets, granulates, biscuits, croquettes, nuggets, flakes and snacks which is also taken into consideration by the present invention.

The finished feed is preferably based on animal or/and plant raw materials. Furthermore, the finished feed preferably contains glycine. The finished feed preferably contains glycine in a ratio to L-serine of more than 1:2.7, preferably of 1:1 or more after hydrolysis.

As already mentioned the finished feed according to the invention is surprisingly stable in storage although it can also have high contents of water. The proposed finished feed should preferably have a water content of >8% by weight, where water contents above 10% by weight and in particular in the range between 20 and 80% by weight are preferred.

The guanidinoacetic acid component according to the invention can, according to the invention, not only be present in a free form i.e. actually as guanidinoacetic acid but also as a salt or in the form of an addition or complex compound. Of course all mixed forms of these compound types are also possible.

Guanidinoacetic acid salts have proven to be favourable for the finished feed according to the invention which are obtained with aspartic acid, ascorbic acid, pyruvic acid, succinic acid, fumaric acid, gluconic acid, oxalic acid, pyroglutamic acid, 3-nicotinic acid, lactic acid, citric acid, maleic acid, sulphuric acid, formic acid, hydrochloric acid and phosphoric acid, where potassium, calcium or sodium guanidinoacetate are particularly suitable. Of course mixtures of guanidinoacetic acid with one or more of the above-mentioned salts can also be used or mixtures which consist of the above-mentioned salts.

As another advantage it has turned out that guanidinoacetic acid and salts thereof can be used in the finished feed in a relatively wide quantity range. Based on the total finished feed it should contain the guanidinoacetic acid component preferably in amounts of 0.01 to 20% by weight, in particular in amounts of 0.1 to 1.0% by weight and particularly preferably in an amount of 0.2 to 0.5% by weight.

Of course in addition to the guanidinoacetic acid component the finished feed can also contain other ingredients such as for example components that are also active with regard to nutritional physiology and/or formulation auxiliaries or fillers.

In this case it may indeed be advisable depending on the respective specific application case to add methyl group donors such as choline, betaine and/or methionine as additional physiologically-active components.

Overall the present invention finds new uses for guanidinoacetic acid and its salts in the diet especially of carnivores such as dogs and cats where they have considerable and surprising advantages compared to the previously known creatine compounds.

The following examples illustrate the breadth of the present invention.

### Examples

### Example 1

A mixture consisting of 5000 mg guanidinoacetic acid and 5000 mg betaine was incorporated in the production of 1 kg of a commercial soft feed for dogs. The amount of guanidinoacetic acid in the final product was 0.5% by weight.

### Example 2

A formulation consisting of 2500 mg guanidinoacetic acid and 5000 mg betaine was incorporated in 1 kg of a typical formulation for canned dog food. The amount of guanidinoacetic acid in the final product was 0.25% by weight.

Example 3

A formulation consisting of 2000 mg guanidinoacetic acid lactate, 750 mg carnitine tartrate, 100 mg sucrose stearate, 160 mg talcum and 1090 mg fructose was incorporated in 1 kg of a base paste for dog biscuits. The amount of guanidinoacetic acid in the final product was 0.2% by weight.

Example 4

The following formulation was incorporated homogeneously in 1 kg of a commercial canned cat food mixture as a master batch: 1000 mg guanidinoacetic acid, 400 mg methionine, 2000 mg choline, 40 mg magnesium stearate, 25 mg carboxymethyl cellulose and 135 mg lactose. The amount of guanidinoacetic acid in the final product was 0.1% by weight.

### Example 5

### Stability

5.1

The stability of creatine and guanidinoacetic acid was compared under conditions which occur when producing industrially manufactured finished feeds. For this purpose a model system was used for the extrusion of a moist feed paste at 160° C. Guanidinoacetic acid and creatine were dissolved in water (pH 7) and heated in an autoclave for 30 minutes to 160° C. Subsequently the content of creatine and guanidinoacetic acid was determined. The rate of the cyclization reaction of creatine to creatinine and of guanidinoacetic acid to glycocyamidine is only dependent on the pH and temperature but completely independent of the concentration.

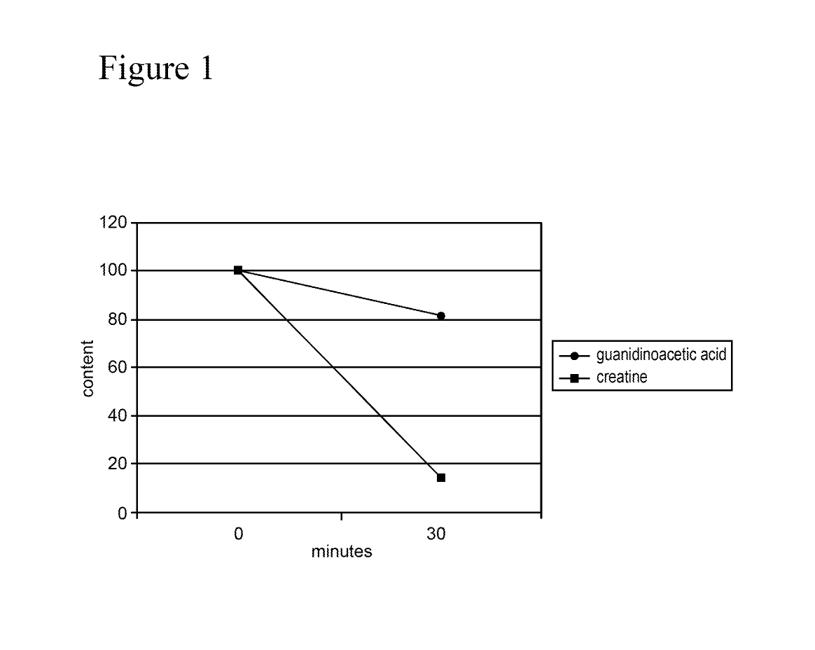

The result of the experiment is shown in FIG. 1. This shows that guanidinoacetic acid has a significantly higher stability than creatine under the conditions of animal feed production. Whereas the creatine content is less than 20% of the original content after 30 minutes at 160° C., more than 80% of the guanidinoacetic acid is still present under the same conditions.

5.2

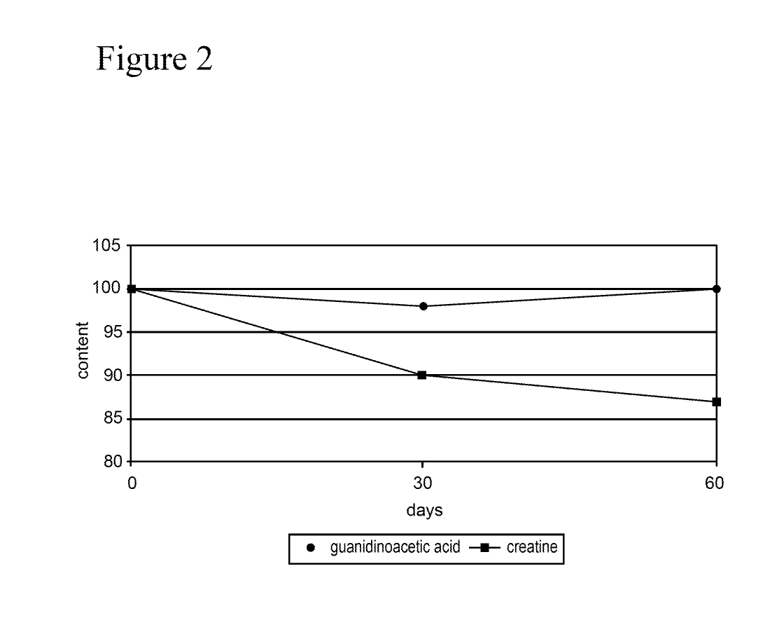

The stability of creatine and guanidinoacetic acid was examined in water at pH 5. These conditions are comparable to storage in canned feeds (75-85% water content). The results are shown in FIG. 2. It can be seen that guanidinoacetic acid has a considerably better storage stability than creatine. Whereas no degradation of guanidinoacetic acid is observed after 60 days, only 87% of the creatine is recovered.

1-9. (canceled)

10. A finished feed for domestic pets comprising at least one guanidinoacetic acid component,
wherein said finished feed is obtained by a process in which said finished feed is industrially processed after addition of the guanidinoacetic acid component to other ingredients of the finished feed,
wherein said finished feed is stable when stored,
and the guanidinoacetic acid component is guanidinoacetic acid in a free form, guanidinoacetic acid as a salt or in a form of an addition or complex compound, which is obtained with guanidinoacetic acid and one or more of malic acid, aspartic acid, ascorbic acid, succinic acid, pyruvic acid, fumaric acid, gluconic acid, a-ketoglutaric acid, oxalic acid, pyroglutamic acid, 3-nicotinic acid, lactic acid, citric acid, maleic acid, sulphuric acid, acetic acid, formic acid, 2-hydroxybenzoic acid, L-carnitine, acetyl-L-carnitine, taurine, betaine, choline, methionine, liponic acid, sodium, potassium or calcium, wherein the guanidinoacetic acid component is present in the finished feed in an amount of 0.01 to 1% by weight.

11. The finished feed of claim 10, wherein the feed is dry feed, semi-moist feed or wet feed.

12. The finished feed of claim 11, wherein the feed is in the form of canned feed, pellets, granulates, biscuits, croquettes, nuggets, flakes or snacks.

13. The finished feed of claim 11, comprising a water content of between 8% and 80% by weight.

14. The finished feed of claim 10, wherein the guanidinoacetic acid component is in a dissolved form.

15. The finished feed of claim 10, further comprising a methyl group donor.

16. The finished feed of claim 18, wherein the methyl donor is choline, betaine, or a combination thereof

17. The finished feed of claim 10, wherein the domestic pet is a carnivore.

18. The finished feed of claim 17, wherein the carnivore is a cat or a dog.

19. A method of providing nutrition to a domestic pet comprising:
administering to the domestic pet a finished feed comprising at least one guanidinoacetic acid component,
wherein said finished feed is obtained by a process in which said finished feed is industrially processed after addition of the guanidinoacetic acid component to other ingredients of the finished feed, wherein said finished feed is stable when stored, and the guanidinoacetic acid component is guanidinoacetic acid in a free form, guanidinoacetic acid as a salt or in a form of an addition or complex compound, which is obtained with guanidinoacetic acid and one or more of malic acid, aspartic acid, ascorbic acid, succinic acid, pyruvic acid, fumaric acid, gluconic acid, α-ketoglutaric acid, oxalic acid, pyroglutamic acid, 3-nicotinic acid, lactic acid, citric acid, maleic acid, sulphuric acid, acetic acid, formic acid, 2-hydroxybenzoic acid, L-carnitine, acetyl-L-carnitine, taurine, betaine, choline, methionine, liponic acid, sodium, potassium or calcium, wherein the guanidinoacetic acid component is present in an amount of 0.01 to 1% by weight.

20. The method of claim 19, wherein the domestic pet is a carnivore.

21. The method of claim 20, wherein the carnivore is a cat or a dog.

22. A method of making a finished feed for domestic pets comprising:
providing ingredients of the finished feed;
obtaining a guanidinoacetic acid component;
mixing the guanidinoacetic acid component with the ingredients to form a mixture; and
industrially processing the mixture to make the finished feed, wherein the guanidinoacetic acid component is present in the finished feed in an amount of 0.01 to 1% by weight.

23. The method of claim 22, wherein said guanidinoacetic acid component is guanidinoacetic acid in a free form, guanidinoacetic acid as a salt or in a form of an addition or complex compound, which is obtained with guanidinoacetic acid and one or more of malic acid, aspartic acid, ascorbic acid, succinic acid, pyruvic acid, fumaric acid, gluconic acid, α-ketoglutaric acid, oxalic acid, pyroglutamic acid, 3-nicotinic acid, lactic acid, citric acid, maleic acid, sulphuric acid, acetic acid, formic acid, 2-hydroxybenzoic acid, L-carnitine, acetyl-L-carnitine, taurine, betaine, choline, methionine, liponic acid, sodium, potassium or calcium.

24. The method of claim 22, wherein industrially processing the mixture comprises heating the mixture to about 160° C. and extruding the heated mixture.

25. A method of providing nutrition to a domestic pet comprising:
administering to the domestic pet a finished feed comprising at least one guanidinoacetic acid component,
wherein said finished feed is obtained by a process in which said finished feed is industrially processed after addition of the guanidinoacetic acid component to other ingredients of the finished feed, wherein said finished feed is stable when stored, and the guanidinoacetic acid component is guanidinoacetic acid in a free form, guanidinoacetic acid as a salt or in a form of an addition or complex compound, which is obtained with guanidinoacetic acid and one or more of malic acid, aspartic acid, ascorbic acid, succinic acid, pyruvic acid, fumaric acid, gluconic acid, α-ketoglutaric acid, oxalic acid, pyroglutamic acid, 3-nicotinic acid, lactic acid, citric acid, maleic acid, sulphuric acid, acetic acid, formic acid, 2-hydroxybenzoic acid, L-carnitine, acetyl-L-carnitine, taurine, betaine, choline, methionine, liponic acid, sodium, potassium or calcium.

26. A method of making a finished feed for domestic pets comprising:
providing ingredients of the finished feed;
obtaining a guanidinoacetic acid component;
mixing the guanidinoacetic acid component with the ingredients to form a mixture; and
industrially processing the mixture to make the finished feed.
